(19) United States
(12) Patent Application Publication
(10) Pub. No.: US 2012/0322651 A1
(43) Pub. Date: Dec. 20, 2012

(54) PROCESS FOR PREPARING METHANOL

(76) Inventors: Holger Schlichting, Hofheim (DE); Philipp Marius Hackel, Usingen (DE); Thomas Wurzel, Oberursel/Ts (DE)

(21) Appl. No.: 13/576,439

(22) PCT Filed: Jan. 28, 2011
(86) PCT No.: PCT/EP2011/000378

§ 371 Date: Aug. 24, 2012

(30) Foreign Application Priority Data
Feb. 22, 2010 (DE) 10 2010 008 857.9

(51) Int. Cl. C07C 29/152; C07C 29/154; B23P 23/00; B01J 37/00

(52) U.S. Cl. 502/100; 518/707; 29/401.1

(57) ABSTRACT

Catalytic process for the preparation of methanol, with a plurality of serial synthesis stages, in which the severity of the reaction conditions, on the basis of the reaction temperature and/or the concentration of carbon monoxide in the synthesis gas, decreases from the first to the last reaction stage in flow direction, the first reaction stage having a first catalyst of low activity but high long-term stability, and the last reaction stage having a second catalyst of high activity but low long-term stability.

This is a 371 of PCT/EP2011/000378 filed 28 Jan. 2011 (international filing date), claiming priority of German application 10 2010 008 857.9 filed 22 Feb. 2010.

The invention relates to a method for the catalytic production of methanol, in which by optimized selection of the catalysts used the economy is distinctly improved as compared to a method known from the prior art. In particular, the invention relates to a method for the optimized methanol synthesis in a multistage process. The invention furthermore relates to a method for converting an existing plant for the production of methanol.

### BACKGROUND OF THE INVENTION

Methods for the production of methanol by catalytic conversion of synthesis gas containing hydrogen and carbon oxides have long since been known to those skilled in the art. For example in Ullmann's Encyclopedia of Industrial Chemistry, Sixth Edition, 1998 Electronic Release, Chapter “Methanol”, Sub-chapter 5.2 “Synthesis”, a single-stage method for the production of methanol is described.

A more advanced, two-stage method for the production of methanol is known for example from the patent specification EP 0 790 226 B1. In this method, a synthesis gas stream containing hydrogen and carbon oxides is reacted in two reaction stages in a water-cooled methanol synthesis reactor, followed by a gas-cooled methanol synthesis reactor. In the last-mentioned reaction stage, the synthesis gas is preheated by indirect heat exchange before entry into the water-cooled methanol synthesis reactor. In both synthesis reactors, usually the same copper-based methanol synthesis catalysts are used. In the described method, the water-cooled reactor typically is operated with a higher synthesis gas inlet temperature than a water-cooled reactor in a single-stage method for methanol synthesis, in order to be able to provide steam with higher pressure. Furthermore, this reactor is charged with synthesis gas that has not been reacted yet. Due the high exothermicity of the methanol synthesis, a very good temperature control of the reactor therefore is necessary, in order to avoid overheating of the catalyst, which would lead to its premature deactivation by loss of active metal surface due to a coagulation of the metal crystallites, the so-called sintering. Beside this thermal effect, it is furthermore known from the prior art that metal/carrier catalysts like the copper-based methanol synthesis catalyst tend to restructure their surface under the influence of carbon monoxide, which leads to the loss of active metal surface by sintering and hence to the loss of activity. As an exemplary reference, the publication of Nihou et al., Journal de Chimie Physique et de Physico-Chimie Biologique (1988), 85(3), pp. 441-448, can be mentioned, in which it could be shown by means of EPMA examinations that the surface of catalysts of the type CuO/ZnO/Al₂O₃ is dynamically restructured during the reaction of carbon oxides with hydrogen under methanol synthesis conditions. At high carbon oxide partial pressures this restructuring is more pronounced than at low carbon oxide partial pressures.

Current developments of catalysts for methanol synthesis, on the other hand, rather aim to provide catalysts which already have a high synthesis activity at low reaction temperatures. In product brochures of methanol synthesis catalysts currently available on the market reference is made to their increased synthesis activity at low temperatures; as an example, the brochure “MK-121—High activity methanol synthesis catalyst” (Haldor Topsoe NS) should be mentioned, which is available on the internet under http://www.topsoe.com/. This increased low-temperature activity becomes possible by an increased dispersion of the copper on the catalyst surface. Developments of other catalyst manufacturers go in the same direction; Süd-Chemie AG, for example, offers the methanol synthesis catalyst MegaMax® 800 as a further development of the catalyst MegaMax® 700, with the first-mentioned, newly developed catalyst being characterized by a higher activity at low temperatures due to an optimized copper dispersion (Nitrogen+Syngas 290, 11-12 (2007), 26-42).

In general, however, metal/carrier catalysts with a high dispersion are more susceptible to deactivation as a result of sintering. Monzón et al. in Applied Catalysis A: General 248 (2003), 279-289, for example could show that the velocity of the decrease in dispersion of noble-metal/carrier catalysts follows a kinetic approach of the kind

dDᵣ/dt=ψ_{D}(Dᵣ−Dᵣᵣ)ⁿ

wherein Dᵣ is the relative dispersion which is defined by the relation Dᵣ=D/D₀, in which D is the absolute value of the dispersion at the time t and D₀ is the absolute value of the dispersion at the time zero. Dᵣᵣ is the limit value of the relative dispersion for t→∞; ψ_{D} is the kinetic constant of the deactivation. n is the kinetic order of the deactivation reaction; in the above-mentioned bibliographical reference it is stated that the same can be described satisfactorily with n=1 to 2 for all data published in the literature on the deactivation kinetics of noble-metal/carrier catalysts due to sintering. Hence it follows that when using a highly dispersed noble-metal/carrier catalyst under otherwise identical conditions, a faster relative decrease in dispersion is to be expected. A faster decrease in dispersion leads to a faster loss of activity and hence to a lower long-term stability of the catalyst. This is particularly disadvantageous in economic terms, since for the newly developed catalyst generations with higher dispersion a higher purchase price mostly is requested on the part of the manufacturers.

Therefore, it is the object of the present invention to avoid the above-mentioned disadvantages and provide a novel, robust, economically advantageous and technically easily feasible method for the production of methanol by maintaining the advantages of the multistage operation.

### SUMMARY OF THE INVENTION

This object substantially is solved with the features of the characterizing part of claim 1 in conjunction with the features of the generic part in that in a method for the catalytic production of methanol from synthesis gas, in which at least two catalyst-containing reaction stages are used with different reaction conditions, in which synthesis gas is at least partly converted to methanol in each, wherein the severity of the reaction conditions as measured by the reaction temperature and/or the concentration of carbon monoxide in the synthesis gas decreases from the first to the last reaction stage in flow direction, a first catalyst with low activity is used in the first reaction stage traversed by synthesis gas and a second catalyst with high activity is used in the last reaction stage traversed by synthesis gas.

From the above-described prior art it is known per se that an opposing relation exists between dispersion or activity and long-term stability of noble-metal/carrier catalysts. What is interesting in the above-described paper of Monzón et al. is the use of the limit value Dᵣᵣ for the relative dispersion, below which the relative dispersion does, however, not decrease any more for defined reaction conditions even with very long operating periods. This means that noble-metal/carrier catalysts which are in operation already for a very long period also have a dispersion and hence residual activity different from zero. Surprisingly, it has now been found that this opposing correlation between the dispersion or activity on the one hand and the long-term stability on the other hand, which has been observed for noble-metal/carrier catalysts, can be transferred to copper-based catalysts for methanol synthesis and leads to a technical teaching which substantially solves the object of the present invention.

### BRIEF DESCRIPTION OF THE DRAWING

FIG. 1 is a schematic illustration of a plant for the production of methanol by the method of the present invention.

### DETAILED DESCRIPTION

According to a preferred aspect of the invention it is provided to use at least one further, third catalyst with medium activity in a method for methanol synthesis with more than two reaction stages. In this way, an optimum adaptation of the catalysts used to the severity of the reaction conditions prevailing in the respective reaction stage is achieved.

An alternative embodiment of the invention provides to use merely two different catalysts with different activity in a method for methanol synthesis with more than two reaction stages. In this way, a slightly worse adaptation of the catalysts used to the severity of the reaction conditions prevailing in the respective reaction stage is achieved as compared to the embodiment described above; however, the limitation to two different types of catalyst leads to logistic advantages and hence in turn to an improved economy of the method.

Advantageously, all catalysts used are copper-based. In virtually all industrial plants operated today, methanol synthesis catalysts of the type Cu/Zn/Al₂O₃ are used for methanol synthesis and are supplied by the trade with different copper dispersions and hence different degrees of activity.

It is particularly advantageous that the at least two reaction stages are integrated into a cycle for non-converted synthesis gas. Even with the highly active methanol synthesis catalysts available today, only a partial conversion of the synthesis gas to methanol is achieved per passage through each reaction stage, so that the recirculation of non-converted synthesis gas to the reaction stages is economically expedient and necessary. In a manner known per se, the cycle operation furthermore serves for temperature control in the reaction stages due to the strongly exothermal reaction.

In a development of the invention, at least one further catalyst-containing reaction stage is arranged in flow direction before the synthesis cycle as pre-reactor for the partial conversion of synthesis gas to methanol, wherein the catalyst has a lower activity than the first reaction stage in flow direction within the synthesis gas cycle. The use of pre-reactors in the methanol synthesis before the synthesis gas cycle is known per se and described for example in the document DE 101 26 719 A1. Since the reaction conditions in the pre-reactor are characterized by a particularly high severity, the use of a methanol synthesis catalyst with low activity, but high long-term stability here offers particularly great advantages.

Furthermore, the method according to the invention is developed in that in flow direction after the synthesis gas cycle at least one further catalyst-containing reaction stage is arranged as post-reactor for the partial conversion of synthesis gas to methanol, wherein the catalyst has a higher activity than the last reaction stage in flow direction within the synthesis gas cycle. Since the synthesis gas entering into the post-reactor already has largely been reacted, the higher activity of the catalyst can optimally be utilized here. Since before charging the synthesis gas to the post-reactor, the previously synthesized methanol is removed by cooling and condensation, the high activity of the methanol synthesis catalyst advantageously can be utilized in that before entry into the post-reactor the synthesis gas must be heated up to temperatures which are lower as compared to the last methanol synthesis reactor within the synthesis cycle, which improves the energy balance of the entire process.

In accordance with a development of the invention, a catalyst of lower activity is not procured from the trade, but such catalyst is provided in that a partly deactivated methanol synthesis catalyst already used in a method for the catalytic methanol synthesis is used as catalyst of lower activity in the method according to the invention. In a particular aspect of this development it is provided to remove the partly deactivated catalyst from the last reaction stage in flow direction, to fill this reaction stage with fresh, highly active catalyst and to use the previously removed, partly deactivated catalyst in an upstream reaction stage, for example in the first reaction stage. For this purpose, it is possibly required to render the partly deactivated catalyst inert to air before removing the same from the last reaction stage in flow direction in a manner known to the skilled person, for example by controlled oxidation, and to reactivate the same after installation in an upstream reaction stage, for example by treatment with reducing gases. With this procedure only fresh, highly active catalyst is procured from the trade, whose period of use can be prolonged according to the invention, whereby further economic advantages are obtained and the amount of deactivated catalyst to be disposed of is reduced.

According to a preferred aspect of the invention two reaction stages are present within the synthesis gas cycle, with the conversion of the synthesis gas initially being effected in a water-cooled reactor and subsequently in a gas-cooled reactor.

The invention furthermore relates to a method for converting an existing plant for the production of methanol from synthesis gas, wherein at least two catalyst-containing reaction stages are used with different reaction conditions, in which synthesis gas each is at least partly converted to methanol, wherein the severity of the reaction conditions as measured by the reaction temperature and/or the concentration of carbon monoxide in the synthesis gas decreases from the first to the last reaction stage in flow direction, which method is characterized in that the catalyst in the first reaction stage traversed by synthesis gas is removed and replaced by a catalyst with low activity. In an alternative embodiment of this method it is provided to leave the aged, partly deactivated catalyst in the water-cooled reactor during the regular shut-down of a method for methanol synthesis with water- and gas-cooled reactor according to the prior art and to replace the likewise aged, partly deactivated catalyst present in the gas-cooled reactor by fresh, highly active catalyst.

Further developments, advantages and possible applications of the invention can also be taken from the following description of exemplary embodiments and the drawings. All features described and/or illustrated form the invention per se or in any combination, independent of their inclusion in the claims or their back-reference.

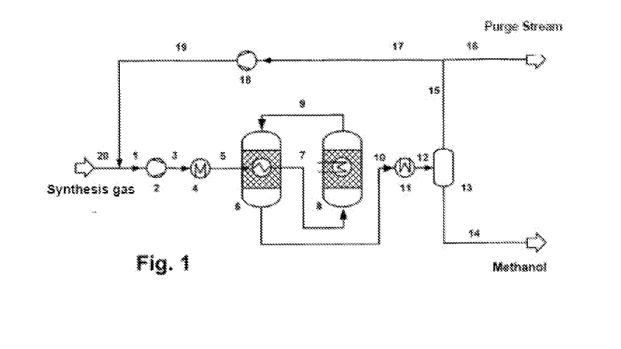

The only FIG. 1 schematically shows a plant for the production of methanol by the method according to the invention. In this FIGURE, a synthesis gas stream containing hydrogen and carbon oxides is supplied via conduit 1 to a compressor 2 and by the same brought to the reaction pressure of typically 5 to 10 MPa. Via conduit 3, the compressed synthesis gas stream is supplied to a heat exchanger 4 and in the same brought to the reaction temperature, wherein the heat exchange mostly is effected against the hot product gas stream from the last synthesis reactor (not shown in FIG. 1). Via conduit 5, the preheated synthesis gas stream enters into the gas-cooled synthesis reactor 6, where it is, however, not yet chemically converted, but initially serves as cooling gas for absorbing the reaction heat released in the reactor 6. At the same time, the cooling gas is heated to reaction temperature up to a temperature of 220 to 280° C. and via conduit 7 then enters into the water-cooled synthesis reactor 8. At temperatures between 200 and 300° C. the partial conversion of hydrogen with carbon oxides is effected here on a methanol synthesis catalyst, wherein a product mixture is obtained, which contains methanol vapor, steam and non-converted synthesis gas. Via conduit 9, the product mixture is discharged from the water-cooled synthesis reactor 8 and charged to the gas-cooled synthesis reactor 6, wherein in the conduit path of conduit 9 a further heat exchanger (not shown in FIG. 1) optionally can be mounted for adapting the temperature of the synthesis gas stream entering into the gas-cooled reactor. At temperatures between 150 and 300° C. the further conversion of hydrogen with carbon oxides is effected here on a methanol synthesis catalyst, wherein a product mixture is obtained, which again contains methanol vapor, steam and non-converted synthesis gas. In the first, water-cooled synthesis reactor a methanol synthesis catalyst of normal activity is used (subsequently also referred to as standard type), whereas in the second, gas-cooled synthesis reactor a highly active methanol synthesis catalyst optimized for lower reaction temperatures is used. The reaction temperature in the second, gas-cooled synthesis reactor therefore lies distinctly below the temperature in the first, water-cooled synthesis reactor, in order to keep the deactivation rate of the highly active methanol synthesis catalyst as low as possible. Alternatively, it can also be equal to the temperature in the first, water-cooled synthesis reactor or even lie above the same, when the CO content of the synthesis gas has been lowered sufficiently by reaction in the first, water-cooled synthesis reactor. Both in the water-cooled and in the gas-cooled synthesis reactor, the space velocity typically amounts to 5000 to 30,000 m³/(m³·h). As explained above, the reaction heat released serves for heating the synthesis gas to the reaction temperature and for generating steam in the water-cooled reactor. The product gas mixture leaves the gas-cooled synthesis reactor via conduit 10. After cooling in the heat exchanger 11, the product gas mixture flows through conduit 12 into the separator 13, where methanol is separated as crude methanol and supplied to the further product processing via conduit 14. Such product processing can be effected in a manner known per se, but not shown in the FIGURE, by distillation or rectification. The gas product obtained in the separator is discharged via conduit 15 and separated into a purge stream, which is discharged via conduit 16, and a cycle stream, which is supplied to the cycle compressor 18 via conduit 17. Via the purge stream, inert components are discharged from the process. Via conduit 19, the cycle stream is recirculated to the synthesis reactor 6, wherein fresh synthesis gas is supplied via conduit 20 and combined with the cycle stream. The ratio of cycle stream to fresh synthesis gas stream is referred to as cycle ratio. It usually lies between 0.5 and 7 m³/m³.

Thus, an economic method for the production of methanol is proposed with the invention, which is characterized in that existing multistage plants for methanol synthesis can continue to be used without any modifications. Due to the lower market price of methanol synthesis reactors of lower activity, economic advantages of the method according to the invention are obtained. Furthermore, with the method according to the invention partly deactivated methanol synthesis catalysts advantageously can continue to be used. The useful life of the catalysts is increased thereby. Furthermore, the amount of catalyst to be disposed of is reduced, which provides advantages in terms of the environmental compatibility of the method according to the invention.

### Numerical Examples

To evaluate the activity and the deactivation behavior of two methanol synthesis catalysts type A (standard type, normal activity) and type B (optimized for high activity at lower temperatures), long-term experiments were carried out in identical, parallel-connected fixed-bed reactors cooled with boiling water. The reactors were charged with synthesis gas of the same composition and the same volumetric flow rate in a straight passage without synthesis gas recirculation. In both fixed-bed reactors, inlet temperature and pressure were the same.

| Catalyst data | Reactor 1 | Reactor 2 |
|---|---|---|
| Catalyst type | Type A | Type B |
| Original form (mm) | 6 × 4 (tablet) | 6 × 4 (tablet) |
| Bulk weight (kg/l) | 1.10 | 1.12 |
| Used volume (ml) | 30.0 | 29.4 |
| Used mass (g) | 32.9 | 32.9 |

The comparative test was carried out with synthesis gas of the composition indicated below.

| CO₂ | 8 | vol % |
|---|---|---|
| CO | 6 | vol % |
| H₂ | 59.5 | vol % |
| CH₄ | 19.5 | vol % |
| N₂ | 7 | vol % |
| Inlet temperature: | 230° C. (270° C. between 630 and 700 h) | |
| Pressure: | 59 | bar |
| Space velocity: | 16,000 | m³/(m³ · h) |

In the following Table, the measured CO turnover is represented for various operating times of the catalysts.

| | CO turnover/% | CO turnover/% |
|---|---|---|
| Operating time/h | Catalyst type A | Catalyst type B |
| 120 | 70 | 68 |
| 440 | 67 | 58 |
| 760 | 61 | 49 |

As shown in the Table, the CO turnovers for catalyst types A and B after 120 h operating time are at a comparable level of 70 and 68%, respectively. After 440 h operating time, the difference between the measured CO turnovers already is 9%, after 760 h operating time even 12%. It should be considered that between 630 and 700 h operating time the reaction temperature has been raised from 230 to 270° C. With the reaction conditions chosen here, as they are typical for the methanol synthesis with methanol synthesis catalysts of the standard type, catalyst type B (optimized for high activity at lower temperatures) quite obviously tends to show a faster loss of activity.

### LIST OF REFERENCE NUMERALS

1 conduit
2 compressor
3 conduit
4 heat exchanger
5 conduit
6 gas-cooled synthesis reactor
7 conduit
8 water-cooled synthesis reactor
9-10 conduit
11 heat exchanger
12 conduit
13 separator
14-17 conduit
18 compressor
19-20 conduit

1. A method for the catalytic production of methanol from synthesis gas, wherein at least two catalyst-containing reaction stages are used with different reaction conditions, in which synthesis gas is at least partly converted to methanol in each, wherein the severity of the reaction conditions as measured by the reaction temperature and/or the concentration of carbon monoxide in the synthesis gas decreases from the first to the last reaction stage in flow direction, wherein a first catalyst is used in the first reaction stage and a second catalyst is used in the last reaction stage said second catalyst having a higher activity than said first catalyst.

2. The method according to claim 1, having more than two reaction stages and at least one further, third catalyst, having medium activity and medium long-term stability, as compared to said first and second catalysts, is used.

3. The method according to claim 1, wherein all catalysts are copper-based.

4. The method according to claim 1, wherein the at least two reaction stages are integrated into a cycle for non-converted synthesis gas.

5. The method according to claim 4, wherein at least one further catalyst-containing reaction stage is arranged in flow direction before the synthesis cycle as pre-reactor for the partial conversion of synthesis gas to methanol, wherein the catalyst has a lower activity than that of the first reaction stage in flow direction within the synthesis gas cycle.

6. The method according to claim 4, wherein at least one further catalyst-containing reaction stage is arranged in flow direction after the synthesis cycle as post-reactor for the partial conversion of synthesis gas to methanol, wherein the catalyst has a higher activity than that of the last reaction stage in flow direction within the synthesis gas cycle.

7. The method according to claim 4, wherein two reaction stages are present within the synthesis gas cycle, and wherein the conversion of the synthesis gas initially is effected in a water-cooled reactor and subsequently in a gas-cooled reactor.

8. Manufacture of catalysts of lower activity, for use in a method according to claim 1, wherein the catalysts are deactivated when they are used for methanol synthesis.

9. (canceled)

10. A method for converting an existing plant for the production of methanol, wherein at least two catalyst-containing reaction stages are used with different reaction conditions, in which synthesis gas is at least partly converted to methanol in each, wherein the severity of the reaction conditions as measured by the reaction temperature and/or the concentration of carbon monoxide in the synthesis gas decreases from the first to the last reaction stage in flow direction, wherein
(a) the catalyst in the first reaction stage traversed by synthesis gas is removed and replaced by a catalyst with lower activity, or
(b) the catalyst in the last reaction stage traversed by synthesis gas is removed and replaced by a catalyst with higher activity, or
(c) both steps (a) and (b) are carried out.

11. The method according to claim 2, wherein all catalysts are copper-based.
